(19) United States
(12) Patent Application Publication
(10) Pub. No.: US 2024/0426809 A1
(43) Pub. Date: Dec. 26, 2024

(54) EVALUATION METHOD OF ANTICANCER DRUG AND SELECTION METHOD OF ANTICANCER DRUG

(71) Applicants: TOPPAN Holdings Inc., Tokyo (JP); JAPANESE FOUNDATION FOR CANCER RESEARCH, Tokyo (JP)

(72) Inventors: Yuki TAKAHASHI, Tokyo (JP); Yuma YOKOKAWA, Tokyo (JP); Ryohei KATAYAMA, Tokyo (JP)

(73) Assignees: TOPPAN Holdings Inc., Tokyo (JP); JAPANESE FOUNDATION FOR CANCER RESEARCH, Tokyo (JP)

(21) Appl. No.: 18/750,552

(22) Filed: Jun. 21, 2024

(30) Foreign Application Priority Data
Jun. 22, 2023 (JP) 2023-102855

(51) Int. Cl. G01N 33/50; C12N 5/077; C12N 5/09

(52) U.S. Cl. CPC G01N 33/5011; C12N 5/0656; C12N 5/0693; C12N 2503/02; C12N 2513/00

(57) ABSTRACT

A evaluation method of an anticancer drug includes culturing a cell structure containing a cancer cell and a stromal cell in a presence of one or two or more of anticancer drugs and evaluating an anticancer effect of the anticancer drug using, as an index, the number of cells having a proliferation ability of the cancer cell in the cell structure after the culturing, in which a signal transduction pathway in the cancer cell stimulated by a hepatocyte growth factor, a placental growth factor, or a vascular endothelial growth factor is more activated than a signal transduction pathway in a normal cell.

### BACKGROUND OF THE INVENTION

### Field of the Invention

The present invention relates to a method of performing a highly reliable evaluation of an anticancer effect of an anticancer drug in an in vitro system without using an animal model and a selection method of an anticancer drug.

This application claims priority to Japanese Patent Application No. 2023-102855, filed Jun. 22, 2023, the entire contents of which are incorporated herein by reference.

### Description of Related Art

For the development of an anticancer drug or an appropriate selection of anticancer drug in cancer treatment, evaluation of the action of an anticancer drug on cancer cells is performed by an in vitro assay system. In addition, the rate of approval of a drug in a domestic pharmaceutical company is extremely low at 0.1%. In order to increase the success rate, it is necessary to early determine whether or not it is probable that the drug candidate substance has a desired drug efficacy, and a highly reliable drug efficacy evaluation method is required. In particular, it has been considered that the limitation of the animal model in the related art is one of the reasons why it is difficult to develop a new drug in a pharmaceutical company. The pharmaceutical company is seeking a drug evaluation model in which an environment in a living body is reproduced more than the animal model.

As an evaluation method of an anticancer drug in an in vitro system, a method of administering an anticancer drug to cancer cells two-dimensionally (that is, planarly) cultured, examining an influence of the anticancer drug on proliferation of the cancer cells, and evaluating the anticancer effect has been performed in the related art. However, even in a case where an anticancer drug has been evaluated to have a high anticancer effect in the evaluation method, an expected anticancer effect is often not obtained in a case where the anticancer drug is actually administered to an animal. Therefore, in the in vitro evaluation method in the related art, because it is not possible to select an appropriate anticancer drug in cancer treatment, and the improvement of the results of cancer treatment is sometimes not achieved.

In the in vitro assay system, it is expected that the anticancer effect in vivo is more accurately reflected by using cells in an environment closer to the in vivo environment. For example, JP2008-011797A describes a method of co-culturing cancer cells and immune cells in a droplet of a collagen gel, and performing an anticancer evaluation of a drug using the obtained spheroid. In addition, it is described that coated cells in which stromal cells are alternately coated with two types of extracellular matrix components is constructed, and a cell structure is constructed by culturing the coated cells after laminating and adhering the coated cells to each other. The cell structure is used in an in vitro assay system for evaluating a drug response (JP5850419B). Furthermore, a cell structure is constructed by collecting a cell mixture in which stromal cells are suspended in a solution containing at least a cationic buffer solution, an extracellular matrix component, and a polymer electrolyte on a substrate and then culturing the cell mixture. The cell structure is used for evaluating an anticancer drug has also been reported (WO2017/183673A).

### SUMMARY OF THE INVENTION

It is considered that a three-dimensional culture method such as a spheroid culture better reproduces an in vivo environment than a two-dimensional culture method in the related art. However, there is no significant difference in sensitivity to a drug such as an anticancer drug between the cell structure obtained by three-dimensional culture and the two-dimensional cultured cells, and there are also not a few examples in which the results are significantly different from those of a cancer-bearing animal model.

An object to be achieved by the present invention is to provide a method of obtaining an evaluation closer to an evaluation obtained in a case of using a cancer-bearing animal model in the drug efficacy evaluation of an anticancer drug by an assay system using cultured cells. As another aspect, a selection method of an anticancer drug in an assay system using cultured cells which can select the anticancer drug based on an evaluation closer to an evaluation obtained in a case of using a cancer-bearing animal model is provided.

As a result of intensive studies to solve the above-described object, the present inventors have found that the sensitivity of cancer cells to anticancer drugs may be affected by a growth factor secreted from a stromal tissue, for example, a hepatocyte growth factor (HGF), a placental growth factor (PIGF), a vascular endothelial growth factor (VEGF), and a basic fibroblast growth factor (bFGF). Therefore, a drug efficacy evaluation for cancer cells in which an intracellular signal transduction pathway downstream of these growth factors is more activated than that in normal cells, which is close to the evaluation obtained in a case of using a cancer-bearing animal model, is obtained by using a three-dimensional cell structure including the cancer cells and stromal cells. As a result, the present invention has been completed.

An evaluation method of an anticancer drug according to a first aspect of the present invention includes culturing a cell structure containing a cancer cell and a stromal cell in a presence of one or two or more of anticancer drugs and evaluating an anticancer effect of the anticancer drug using, as an index, the number of cells having a proliferation ability of the cancer cells in the cell structure after the culturing, in which a signal transduction pathway in the cancer cells stimulated by a hepatocyte growth factor, a placental growth factor, a vascular endothelial growth factor, or a basic fibroblast growth factor is more activated than a signal transduction pathway in a normal cell.

As another aspect, a selection method of an anticancer drug according to a second aspect of the present invention includes culturing a cell structure containing a cancer cell and a stromal cell in a presence of one or two or more of anticancer drugs and selecting an anticancer drug as an anticancer drug having an anticancer effect to the cancer cells when, using the number of cells having a proliferation ability of the cancer cells in the cell structure after the culturing as an index, the number of cells having a proliferation ability of the cancer cells in the cell structure is less than the case where the cell structure is cultured under the condition where the anticancer drug does not exist, in which a signal transduction pathway in the cancer cells stimulated by a hepatocyte growth factor, a placental growth factor, a vascular endothelial growth factor, or a basic fibroblast growth factor is more activated than a signal transduction pathway in a normal cell.

In the first and second aspects of the present invention, the signal transduction pathway is preferably an ERK/MAPK pathway, a PI3K/AKT pathway, a Jak-STAT pathway, or a WNT/β-Catenin pathway.

In the first and second aspects of the present invention, the cancer cell is preferably one or more selected from the group consisting of a HER2-positive cancer cell, an ALK-positive cancer cell, a BRAF-positive cancer cell, and a ROS1-positive cancer cell.

In the first and second aspects of the present invention, the anticancer drug preferably targets a molecule constituting a signal transduction pathway stimulated by a hepatocyte growth factor, a placental growth factor, or a vascular endothelial growth factor, or a molecule activating the molecule.

In the first and second aspects of the present invention, the anticancer drug is preferably a HER2 inhibitor, an ALK inhibitor, a BRAF inhibitor, or a WNT inhibitor.

In the first and second aspects of the present invention, the stromal cell is preferably one or more selected from the group consisting of a fibroblast, a dendritic cell, a macrophage, and a mast cell.

In the first and second aspects of the present invention, the cell structure preferably has a cancer cell layer on an outer surface.

In the first and second aspects of the present invention, the cell structure may include, as a cell constituting the stroma, a fibroblast and one or more selected from the group consisting of a vascular endothelial cell and a lymphatic endothelial cell, and a total number of cells of the vascular endothelial cell and the lymphatic endothelial cell in the cell structure may be 0.1% or more of a number of cells of the fibroblast.

In the first and second aspects of the present invention, a thickness of the cell structure may be 5 m or more.

In the first and second aspects of the present invention, the cell structure may include a vascular network structure.

In the first and second aspects of the present invention, the cancer cell may be a cancer cell collected from a cancer patient.

In the first and second aspects of the present invention, a culture time in the culture step may be 24 to 96 hours.

In the first and second aspects aspect of the present invention, before the culture step, forming the cell structure may be further included, the forming the cell structure may be (a) mixing cells, an extracellular matrix component, and a polymer electrolyte in a cationic buffer solution to obtain a mixture, (b) seeding the mixture obtained in the (a) in a cell culture container, and (c) obtaining a cell structure in which the cells are laminated in multilayers in the cell culture container after the (b).

### BRIEF DESCRIPTION OF THE DRAWINGS

FIG. 1 is a diagram showing a sensitivity evaluation result of a non-small cell lung cancer cell LCC007 strain to Lapatinib in Example 1. (A) of FIG. 1 shows the results of measuring the relative cell survival rate (%) of each cell group over time, and (B) of FIG. 1 shows the results of measuring the tumor volume of each mouse group over time.

FIG. 2 is a diagram showing a sensitivity evaluation result of a non-small cell lung cancer cell LCC381 strain to Lapatinib in Example 1. (A) of FIG. 2 shows the results of measuring the relative cell survival rate (%) of each cell group over time, and (B) of FIG. 2 shows the results of measuring the tumor volume of each mouse group over time.

FIG. 3A is a diagram showing the results of measuring the amount of HGF in a culture supernatant of NHDF and HUVEC 2D-cultured and a culture supernatant of NHDF and HUVEC 3D-cultured, in Example 2.

FIG. 3B is a diagram showing the results of measuring the amount of PIGF in a culture supernatant of NHDF and HUVEC 2D-cultured and a culture supernatant of NHDF and HUVEC 3D-cultured, in Example 2.

FIG. 3C is a diagram showing the results of measuring the amount of VEGF in a culture supernatant of NHDF and HUVEC 2D-cultured and a culture supernatant of NHDF and HUVEC 3D-cultured, in Example 2.

FIG. 3D is a diagram showing the results of measuring the amount of bFGF in a culture supernatant of NHDF and HUVEC 2D-cultured and a culture supernatant of NHDF and HUVEC 3D-cultured, in Example 2.

FIG. 4 is an image of western blotting performed to examine an activation state of a molecule in a signal transduction pathway stimulated by HGF in 2D-cultured cells of non-small cell lung cancer cell LCC007 strain, a cell structure obtained by 3D-culturing the LCC007 cell line with NHDF and HUVEC, and a tumor tissue in a cancer-bearing mouse into which the LCC007 strain has been transplanted, in Example 3.

FIG. 5A is a diagram showing a sensitivity evaluation result of a non-small cell lung cancer cell LCC028-3 strain to an ALK inhibitor in Example 4.

FIG. 5B is a diagram showing a sensitivity evaluation result of a colon cancer cell JC215 strain to a BRAF inhibitor in Example 4.

FIG. 6A is a diagram showing a sensitivity evaluation result of a colon cancer cell JC215 strain to a WNT inhibitor in Example 4.

FIG. 6B is a diagram showing a sensitivity evaluation result (FIG. 6B) of a non-small cell lung cancer cell LCC168 strain in Example 4.

### PREFERRED EMBODIMENTS

In the present embodiment and herein, the “cell structure” is a three-dimensional structure in which a plurality of cell layers are laminated. The “cell layer” is a layer constituted of a stroma and a group of cells that are present in a direction orthogonal to the thickness direction and that are present without overlapping with each other in the thickness direction, in a case of being observed at a magnification at which a cell nucleus can be recognized, that is, at a magnification at which the entire thickness of a stained section is within a field of view in a section image of the cell structure in the thickness direction. In addition, the “layered” means that different cell layers are laminated in a thickness direction in two or more layers.

In the present specification and herein, “cell aggregate” means a group of cells. The cell aggregate also includes a precipitate of cells (an aggregate of cells formed by precipitating cells) obtained by centrifugation, filtration, or the like. In a certain embodiment, the cell aggregate is a slurry-like viscous body. In the present specification, the “slurry-like viscous body” refers to a gel-like cell aggregate as described in Akihiro Nishiguchi et al., Macromol Biosci. 2015 March; 15(3): 312-7.

An evaluation method of an anticancer drug according to a first embodiment of the present invention will be described.

The evaluation method of an anticancer drug according to the first embodiment of the present invention (hereinafter, may be referred to as “evaluation method according to the present embodiment”) includes a culture step of culturing a cell structure containing cancer cells and cells (stromal cells) constituting a stroma in the presence of one or two or more of anticancer drugs, and an evaluation step of evaluating an anticancer effect of the anticancer drug using, as an index, the number of cells having a proliferation ability in the cancer cells in the cell structure after the culture step.

The evaluation method according to the present embodiment includes evaluating an anticancer effect using a cell structure containing cancer cells and cells (hereinafter, may be simply referred to as stromal cells) constituting a stroma (hereinafter, may be referred to as “cell structure according to the present embodiment”). The cell structure according to the present embodiment is a cell structure containing the cancer cell for the purpose of evaluating a drug efficacy of the anticancer drug on an inside or a surface of a three-dimensional structure including at least stromal cells. The stroma is an important configuration in a cancer microenvironment in a living body. In particular, cancer cells in a living body interact with surrounding stromal cells to form a microenvironment, and the microenvironment affects the sensitivity to an anticancer drug. That is, by coexisting the cancer cells in the cell structure (hereinafter, may be referred to as laminated artificial stromal tissue) having a three-dimensional structure formed by laminating the stromal cells, the environment of the cancer cells in the living body is reproduced, and the anticancer action in the living body can be appropriately evaluated. Therefore, by using the cell structure, the evaluation of an anticancer drug that more reflects the clinical results of a human even in an in vitro evaluation system can be performed, and a highly reliable evaluation can be obtained.

In the cell structure according to the present embodiment, since the stromal cells are formed as a three-dimensional structure such as the stromal tissue in the living body, various components are secreted as in the stromal tissue in the living body. The drug sensitivity of the cancer cell is affected by the secreted material, but the influence depends on the type of the cancer cell and the drug, namely, the anticancer drug. Since the stromal cells in the cell structure according to the present embodiment secrete HGF, PIGF, VEGF, and bFGF, the cancer cells in the cell structure are under the influence of these growth factors, similarly to the cancer cells in the stromal tissue in the living body. Therefore, the drug sensitivity of the cancer cell in the cell structure according to the present embodiment is close to the drug sensitivity of the cancer cell in the living body, and it is possible to obtain a drug efficacy evaluation close to the evaluation obtained in a case of using the cancer-bearing animal model by the evaluation method according to the present embodiment.

The evaluation method according to the present embodiment is particularly preferably applied to a cancer cell in which a signal transduction pathway stimulated by at least one of HGF, PIGF, VEGF, and bFGF is activated than in a normal cell. That is, the cancer cell contained in the cell structure according to the present embodiment is preferably a cancer cell in which a signal transduction pathway stimulated by at least one of HGF, PIGF, VEGF, and bFGF is more activated than in a normal cell. In the cancer cell in which the signal transduction pathway stimulated by these growth factors is activated, the sensitivity to an anticancer drug is significantly different in the presence and absence of these growth factors. For example, in a cancer cell in which activation of a signal transduction pathway causes resistance (namely, decrease of the sensitivity) to an anticancer drug, the sensitivity to the anticancer drug is decreased in the presence of a growth factor that stimulates the signal transduction pathway as compared with in the absence of the growth factor. On the contrary, in the cancer cell in which the activation of the signal transduction pathway causes the sensitization (namely, enhancement of the sensitivity) to the anticancer drug, the sensitivity to the anticancer drug is increased in the presence of the growth factor that stimulates the signal transduction pathway as compared with in the absence of the growth factor. Therefore, in the evaluation in an in vitro assay system in which these growth factors are insufficient, the deviation from the evaluation in a case of using an animal model is very large. By using the cell structure according to the present embodiment, it is possible to obtain a highly reliable drug efficacy evaluation even for such cancer cells.

The signal transduction pathway stimulated by HGF, PIGF, VEGF, or bFGF is not particularly limited. In addition, in a case where there are two or more types of signal transduction pathways stimulated by one type of growth factor, it is sufficient that at least one type of signal transduction pathway is activated. For example, HGF is a ligand for a hepatocyte growth factor receptor (MET), and an ERK/MAPK pathway, a PI3K/AKT pathway, or a WNT/β-Catenin pathway in a cell is activated by MET activated by binding to HGF (Raghav et al, Translational Lung Cancer Research, 2012, vol. 1(3), p. 179 to 193). In addition, VEGF is a ligand for a vascular endothelial cell proliferation factor receptor (VEGFR), and a Jak-STAT pathway in a cell is activated by VEGFR activated by binding to VEGF (Owen et al, Cancers, 2019, vol. 11, 2002).

Whether or not the signal transduction pathway stimulated by HGF, PIGF, VEGF, or bFGF is activated can be determined, for example, by using, as an index, the abundance of an activated molecule (for example, a phosphorylated molecule, a molecule having a gene mutation, or the like) in a cell for any molecule constituting the signal transduction pathway.

Examples of the molecule activated by phosphorylation among the molecules constituting the signal transduction pathway include ERK, MAPK, and the like in the ERK/MAPK pathway. Examples thereof include Akt, S6, and the like in the PI3K/AKT pathway. Examples thereof include Jak, STAT, and the like in the Jak-STAT pathway.

For a molecule A activated by phosphorylation among the molecules constituting the signal transduction pathway, in a case where a phosphorylated molecule pA of the molecule A is not detected in a normal cell but the phosphorylated molecule pA is detected in a cancer cell, it can be said that the cancer cell is a cancer cell in which the signal transduction pathway is activated. In addition, also in a case where [amount (relative value) of phosphorylated molecules pA in the cancer cell]/[amount (relative value) of phosphorylated molecules pA in the normal cell] is 1.1 or more, preferably 1.2 or more, and more preferably 1.5 or more, it can be said that the cancer cell is a cancer cell in which the signal transduction pathway is activated. The amount of the phosphorylated molecule can be measured by a known method such as a Western blotting method using an antibody that specifically recognizes the molecule.

Examples of the molecule that is constantly activated by gene mutation among the molecules constituting the signal transduction pathway include Ras mutation, Raf mutation, and the like in the ERK/MAPK pathway. Examples thereof include Akt mutation, PI3K mutation, and PTEN mutation in the PI3K/AKT pathway. Examples thereof include β-catenin, Wnt, and the like in the WNT/β-catenin pathway. Examples thereof include Jak, STAT, and the like in the Jak-STAT pathway.

Specific examples of the cancer cell in which the signal transduction pathway stimulated by HGF, PIGF, VEGF, or bFGF is activated include a HER2-positive cancer cell (namely, a cancer cell in which the expression level of HER2 is increased because of a gene abnormality of the HER2 gene), an ALK-positive cancer cell (namely, a cancer cell having an ALK fusion gene), a BRAF-positive cancer cell (namely, a cancer cell having a gene mutation of the BRAF gene), and a ROS1-positive cancer cell (namely, a cancer cell having a ROS1 fusion gene). In the evaluation method according to the present embodiment, it is preferable to evaluate the sensitivity of these cancer cell to one or two or more anticancer drugs.

### <Cell Structure>

The cell structure (cell structure according to the present embodiment) used in the present embodiment includes at least a cancer cell and a stromal cell. The stromal cell contained in the cell structure according to the present embodiment may be one type or two or more types. The cancer cell contained in the cell structure according to the present embodiment may be one type or two or more types. The cancer cell is a cell that has been derived from a somatic cell and has acquired an infinite proliferation ability.

The cell containing a stromal cell and a cancer cell which constitutes the cell structure according to the present embodiment are not particularly limited, and the cell may be a cell collected from an animal, may be a cell obtained by culturing a cell collected from an animal, may be a cell obtained by subjecting a cell collected from an animal to various treatments, or may be a cultured cell strain. In a case of a cell collected from an animal, a collection site is not particularly limited, and the cell may be a somatic cell derived from a bone, a muscle, an internal organ, a nerve, a brain, a bone, a skin, blood, or the like, may be a germ cell, or may be an embryonic stem cell (ES cell). In addition, the biological species from which the cell constituting the cell structure according to the present embodiment is derived is not particularly limited, and for example, cells derived from animals such as human, monkey, dog, cat, rabbit, pig, cow, mouse, and rat can be used. The cell obtained by culturing a cell collected from an animal may be a primary cultured cell or may be a subcultured cell. In addition, examples of the cell that has been subjected to various treatments include an induced pluripotent stem cell (iPS cell) and a cell after differentiation induction. Furthermore, the cell structure according to the present embodiment may be constituted of only cells derived from the same biological species, or may be constituted of cells derived from a plurality of biological species.

Examples of the stromal cells include endothelial cells, fibroblasts, nerve cells, mast cells, epithelial cells, myocardial cells, hepatocytes, islet cells, tissue stem cells, smooth muscle cells, and the like. The stromal cell contained in the cell structure according to the present embodiment may be one type or two or more types. The cell type of the stromal cell contained in the cell structure according to the present embodiment is not particularly limited, and can be appropriately selected in consideration of the origin and type of the cancer cell to be contained, the type of the anticancer drug to be used for evaluation, the environment in a living body in which the desired anticancer activity is exhibited, and the like.

The blood vessel network structure and the lymphatic vessel network structure are important for the proliferation and the activity of the cancer cells. Therefore, the cell structure according to the present embodiment preferably includes a vascular network structure. That is, the cell structure according to the present embodiment is preferably a cell structure in which inside a laminate of cells not forming a vessel, a vascular network structure such as a lymphatic vessel and/or a blood vessel is three-dimensionally constructed and a tissue closer to a tissue in the living body is constructed. The vascular network structure may be formed only inside the cell structure, or may be formed such that at least a part of the vascular network structure is exposed on the surface or the bottom surface of the cell structure. In the present embodiment and herein, the “vascular network structure” refers to a network-like structure such as a blood vessel network or a lymphatic vessel network in a biological tissue.

The vascular network structure can be formed by including endothelial cells constituting vessels as stromal cells. The endothelial cell contained in the cell structure according to the present embodiment may be a vascular endothelial cell or a lymphatic endothelial cell. In addition, both the vascular endothelial cell and the lymphatic endothelial cell may be included.

In a case where the cell structure according to the present embodiment includes a vascular network structure, as the cells other than the endothelial cells in the cell structure, the cells constituting a peripheral tissue of a vessel in a living body are preferable since the endothelial cells easily form a vascular network that retains the original function and shape. The cells including at least fibroblasts as the cells other than the endothelial cells are more preferable since the cells are more approximated to a cancer microenvironment in a living body, and cells including vascular endothelial cells and fibroblasts, cells including lymphatic endothelial cells and fibroblasts, or cells including vascular endothelial cells, lymphatic endothelial cells, and fibroblasts are still more preferable. The cells other than the endothelial cells contained in the cell structure may be a cell derived from the same biological species as the endothelial cell or may be a cell derived from a different biological species.

The number of endothelial cells in the cell structure according to the present embodiment is not particularly limited as long as a sufficient number of endothelial cells are present to form a vascular network structure, and can be appropriately determined in consideration of the size of the cell structure, the cell type of the endothelial cells or cells other than the endothelial cells, and the like. For example, by setting the presence ratio (number ratio of cells) of the endothelial cells to the total cells constituting the cell structure according to the present embodiment to 0.1% or more, a cell structure in which a vascular network structure is formed can be prepared. In a case where fibroblasts are used as the cells other than the endothelial cells, the number of endothelial cells in the cell structure according to the present embodiment is preferably 0.1% or more and more preferably 0.1% to 5.0% of the number of fibroblasts. In a case where both the vascular endothelial cell and the lymphatic endothelial cell are included as the endothelial cell, the total number of the vascular endothelial cells and the lymphatic endothelial cells is preferably 0.1% or more and more preferably 0.1% to 5.0% of the number of the fibroblasts.

The number of cancer cells in the cell structure according to the present embodiment is not particularly limited, but a ratio ([number of endothelial cells]/[number of cancer cells]) of the number of endothelial cells to the number of cancer cells in the cell structure is preferably more than 0 (larger than 0) and 1.5 or less since the cell structure is made to approximate the cancer microenvironment in the living body. In addition, in a case where the cell structure containing the endothelial cells, the fibroblasts, and the cancer cells is used, a ratio ([number of fibroblasts]/[number of cancer cells]) of the number of fibroblasts to the number of cancer cells in the cell structure is preferably 0.6 to 100 and more preferably 50 to 100.

The total number of cells constituting the cell structure according to the present invention is not particularly limited, but from the viewpoints that a structure in which a layer formed of the vascular endothelial cells includes other cell populations is easily formed and that the formed cell structure is easily observed, the total number of cells is preferably a number of cells corresponding to 15 to 25 theoretical cell layers. The theoretical number of cell layers is represented by the following expression (1). In the expression (1), “N_{A}” represents the total number of cells constituting the cell structure, “N_{S}” represents the number of cells per layer in a case where the cell structure is constructed of the same type of cells on the same type of substrate, and “L” represents the theoretical number of cell layers.

[L]=[N_{A}]/[N_{S}] (1)

N_{S} can be experimentally obtained. For example, in a case where a cell suspension is prepared using 96-well plate as a substrate, 0.9×10⁶ pieces of NHDFs, and 0.0135×10⁶ pieces of HUVECs, in the constructed cell structure, about 20 nuclei are vertically laminated. Therefore, in a case of a cell structure constructed using a 96-well plate as a substrate, NHDF, and HUVEC, N_{A} is set to 0.45×10⁵ pieces.

The cancer cell contained in the cell structure according to the present embodiment may be a strained cultured cell or may be a cancer cell collected from a cancer patient. The cancer cell collected from the cancer patient may be a cell that has been cultured in advance and proliferated. Specific examples of the cancer cell include primary cancer cells collected from a cancer patient, artificially cultured cancer cells, iPS cancer stem cells, cancer stem cells, strained cancer cells that are prepared in advance for use in research on cancer treatment or development of anticancer drugs, and the like. In addition, the cancer cell may be a cancer cell derived from human or a cancer cell derived from an animal other than a human. In a case where the cell structure according to the present embodiment contains a cancer cell collected from a cancer patient, the cell structure may also contain a cell other than the cancer cell collected from the cancer patient, together with the cancer cell. Examples of the cell other than the cancer cell include one or more types of cells contained in a solid tissue excised after surgery.

Examples of cancers from in which the cancer cell contained in the cell structure according to the present embodiment is derived include breast cancer (such as infiltrating ductal carcinoma, ductal carcinoma in situ, and inflammatory breast cancer), prostate cancer (such as hormone-dependent prostate cancer and hormone-independent prostate cancer), pancreatic cancer (such as pancreatic duct cancer), gastric cancer (such as papillary adenocarcinoma, mucinous adenocarcinoma, and adenosquamous carcinoma), lung cancer (such as non-small cell lung cancer, small cell lung cancer, and malignant mesothelioma), colon cancer (such as gastrointestinal stromal tumor), rectal cancer (such as gastrointestinal stromal tumor), colorectal cancer (such as familial colorectal cancer, hereditary nonpolyposis colorectal cancer, and gastrointestinal stromal tumor), small intestine cancer (such as non-Hodgkin lymphoma and gastrointestinal stromal tumor), esophageal cancer, duodenal cancer, tongue cancer, pharyngeal cancer (such as nasopharyngeal cancer, oropharyngeal cancer, and hypopharyngeal cancer), head and neck cancer, salivary grand cancer, brain tumors (such as pineal astrocytoma, pilocytic astrocytoma, diffuse astrocytoma, and anaplastic astrocytoma), neurinoma, liver cancer (such as primary liver cancer, extrahepatic bile duct cancer), kidney cancer (such as renal cell cancer, transitional cell carcinoma of the renal pelvis and ureter), gallbladder cancer, bile duct cancer, pancreatic cancer, hepatoma, endometrial cancer, cervical cancer, ovarian cancer (such as epithelial ovarian cancer, extragonal germ cell tumor, ovarian embryonic cell tumor, or low-grade ovarian tumor), bladder cancer, urethral cancer, skin cancer (such as intraocular (ocular) melanoma, and Merkel cell cancer), hemangioma, malignant lymphoma (such as reticular sarcoma, lymphosarcoma, and Hodgkin's disease), melanoma (malignant melanoma), thyroid cancer (such as thyroid medullary carcinoma), parathyroid cancer, nasal cavity cancer, sinus cancer, bone tumors (such as osteosarcoma, Ewing's tumor, uterine sarcoma, and soft tissue sarcoma), metastatic medulloblastoma, hemangiofibroma, dermatofibrosarcoma protuberans, retinal sarcoma, penile cancer, testicular tumor, pediatric solid cancer (such as Wilms tumor and pediatric renal tumor), Kaposi's sarcoma, Kaposi's sarcoma caused by AIDS, maxillary sinus tumors, fibrous histiocytoma, leiomyosarcoma, rhabdomyoblastoma, chronic myeloproliferative disease, and leukemia (such as acute myeloid leukemia and acute lymphoblastic leukemia), and the cancer is not limited thereto.

The cell structure according to the present embodiment may be a cell structure in which the cancer cells are scattered throughout the entire inside of the structure, or may be a cell structure in which the cancer cells are present only in a specific cell layer. In addition, the stromal cell layer and the cancer cell layer may be a cell structure that is partitioned by a semi-permeable membrane.

In the cell structure according to the present embodiment, in a case where the cancer cell is present only in a specific cell layer, a position of the cell layer (hereinafter, may be referred to as cancer cell layer) containing the cancer cell in the cell structure is not particularly limited. Since the influence of the immune cells and/or the anticancer drug can sufficiently reach, a position in a thickness direction of the cancer cell layer in the cell structure is preferably within a range from a top surface (namely, upper surface) of the structure to a half height in the thickness direction. In particular, in a case where the cell structure is cultured in the presence of immune cells, by providing the cancer cell layer inside the cell structure instead of the top surface of the cell structure, it is possible to evaluate the anticancer effect including the ability of the immune cells to infiltrate and/or reach the cancer cells in the cell structure. In the present specification, the thickness of the cell structure is a length in a self-weight direction of the tissue. The self-weight direction is a direction in which gravity is applied, and is also referred to as a thickness direction.

The cell structure according to the present embodiment may contain cells other than the cancer cell and the stromal cell. Examples of the other cells include immune cells, nerve cells, hepatocytes, pancreatic cells, myocardial cells, smooth muscle cells, bone cells, alveolar epithelial cells, spleen cells, and the like.

A size and a shape of the cell structure according to the present embodiment are not particularly limited. Since a vascular network structure in a state close to a vessel formed in a tissue in a living body can be formed and more accurate evaluation is possible, the thickness of the cell structure is preferably 5 m or more, more preferably m or more, still more preferably 50 m or more, and even still more preferably 100 m or more. In addition, the thickness of the cell structure is preferably 500 m or less, more preferably 400 m or less, and still more preferably 300 m or less. The upper limit value and the lower limit value of the thickness of the cell structure can be arbitrarily combined. The number of the cell layers in the cell structure according to the present embodiment is preferably about 2 to 60 layers, more preferably about 5 to 60 layers, and still more preferably about 10 to 60 layers.

The number of cell layers constituting the cell structure is measured by dividing the total number of cells constituting the three-dimensional structure by the number of cells per layer (the number of cells required to constitute one layer). The number of cells per layer can be examined by planarly culturing cells confluently in advance in a cell culture container used in the case of constituting the cell structure. Specifically, the number of cell layers in the cell structure formed in a certain cell culture container can be calculated by measuring the total number of cells constituting the cell structure and dividing the total number of cells by the number of cells per layer in the cell culture container.

In general, the cell structure according to the present embodiment is constructed in a cell culture container. The cell culture container is not particularly limited as long as it is a container capable of constructing a cell structure and capable of culturing the constructed cell structure. Specific examples of the cell culture container include a dish, a cell culture insert (for example, a Transwell (registered trademark) insert, a Netwell (registered trademark) insert, a Falcon (registered trademark) cell culture insert, a Millicell (registered trademark) cell culture insert, and the like), a tube, a flask, a bottle, a plate, and the like. In the construction of the cell structure according to the present embodiment, a dish or various cell culture inserts is preferable since the evaluation using the cell structure can be more appropriately performed.

The cell structure according to the present embodiment need only be a structure formed of a multilayered cell layer containing cancer cells and stromal cells, and a method of constructing the cell structure is not particularly limited. For example, a method of constructing the cell layer by constructing the cell layer one by one and sequentially laminating the cell layers may be used, a method of constructing two or more cell layers at once may be used, or a method of constructing a multilayered cell layer by appropriately combining both construction methods may be used. In addition, the cell structure according to the present embodiment may be a multilayered structure in which the cell type constituting each cell layer is different for each layer, and the cell type constituting each cell layer may be a cell type common to all the layers of the structure. For example, a method of constructing the cell structure by forming a layer for each cell type and sequentially laminating the cell layers for each cell type may be used, or a method of preparing a cell mixture solution obtained by mixing a plurality of types of cells in advance and constructing the cell structure of the multilayered structure at once from the cell mixture solution in which the plurality of types of cells are mixed, which is prepared in advance, may be used.

Examples of the method of constructing the cell layer by constructing the cell layer one by one and sequentially laminating the cell layers include the method described in JP4919464B, that is, a method of continuously laminating the cell layers by alternately repeating a step of forming a cell layer and a step of bringing the formed cell layer into contact with a solution containing a component of an extracellular matrix (ECM). For example, in a case of performing the method, a cell structure in which a vascular network structure is formed in the entire structure and cancer cells are scattered in the entire structure can be constructed by preparing a cell mixture in which all the cells constituting the cell structure are mixed in advance, and forming each cell layer with this cell mixture. In addition, by forming each cell layer for each cell type, a cell structure in which a vascular network structure is formed only in a layer formed from endothelial cells and cancer cells are present only in a specific cell layer can be constructed.

Examples of the method of constructing two or more cell layers at once include the method described in JP5850419B. The method is a method of constructing a cell structure formed of a multi-layered cell layer, coating the entire surface of a cell in advance with a polymer containing an arginine-glycine-aspartic acid (RGD) sequence to which an integrin binds and a polymer which interacts with the polymer containing the RGD sequence, accommodating the coated cells coated with the adhesive membrane in a cell culture container, and then accumulating the coated cells by centrifugation or the like. For example, in a case of performing the method, a cell mixture in which all the cells constituting the cell structure are mixed is prepared in advance, and a coated cell prepared by adding the adhesive component to this cell mixture is used. As a result, a cell structure in which cancer cells are scattered throughout the entire structure can be constructed by one centrifugal treatment. In addition, for example, a coated cell in which endothelial cells are coated, a coated cell in which fibroblasts are coated, and a coated cell in which a cell group collected from a cancer patient is coated are each separately prepared, a multilayer constituted of coated cells of fibroblasts is formed, then one layer constituted of coated cells of endothelial cells is laminated thereon, a multilayer constituted of coated cells of fibroblasts is further laminated thereon, and one layer constituted of coated cells of a cell including a cancer cell is further laminated thereon.

As a result, a cell structure can be constructed, which includes a vascular network structure interposed between a thick fibroblast layer and includes a layer containing cancer cells collected from a cancer patient on the top surface.

The cell structure according to the present embodiment can also be constructed by a method including the following steps (a) to (c).
(a) a step of mixing a cell with an extracellular matrix component in a cationic buffer solution to obtain a mixture, (b) a step of seeding the mixture obtained in the step (a) in a cell culture container, and (c) a step of obtaining a cell structure in which cells are laminated in a multilayer in the cell culture container after the step (b).

In the step (a), by mixing the cells with a buffer solution containing a cationic substance (cationic buffer solution) and an extracellular matrix component to form a cell aggregate from the cell mixture, it is possible to obtain the cell aggregate as a three-dimensional cell tissue having few large voids inside. In addition, since the obtained three-dimensional cell tissue is relatively stable, culture for at least several days is capable and the tissue is less likely to be destroyed also in a case of exchanging the culture medium. In addition, in the present embodiment, in the step (b), the precipitation of the cell mixture seeded in the cell culture container in the cell culture container can be included. The precipitation of the cell mixture may be actively performed by precipitating the cells by centrifugal separation or the like, or may be performed by natural precipitation.

As the cationic substance used in the present embodiment, any substance having a positive charge can be used as long as it does not have a bad influence on the growth of cells and the formation of cell aggregates. Examples of the cationic substance include a cationic buffer solution such as a tris-hydrochloric acid buffer solution, a tris-maleic acid buffer solution, a bis-tris buffer solution, and HEPES, ethanolamine, diethanolamine, triethanolamine, polyvinylamine, polyallylamine, polylysine, polyhistidine, polyarginine, and the like.

The concentration and pH of the cationic substance (for example, tris in the tris-hydrochloric acid buffer solution) in the cationic buffer solution are not particularly limited as long as the growth of the cells and the construction of the cell structure are not adversely affected. For example, the concentration of the cationic substance in the cationic buffer solution can be set to 10 to 100 mM, preferably 40 to 70 mM and more preferably 50 mM. In addition, the pH of the cationic buffer solution can be set to 6.0 to 8.0, preferably 6.8 to 7.8 and more preferably 7.2 to 7.6.

In the step (a), it is preferable to further mix the cells with a polymer electrolyte. By mixing the cells with the cationic substance, the polymer electrolyte, and the extracellular matrix component, a three-dimensional cell tissue having few voids and a large thickness can be obtained even in a case of natural precipitation without a treatment of positively aggregating the cells such as centrifugation in the step (b).

In the present invention and herein, the “polymer electrolyte” means a polymer having a functional group which can be dissociated in a polymer chain. As the polymer electrolyte used in the present embodiment, any polymer electrolyte can be used as long as the polymer electrolyte does not adversely affect the growth of cells and the formation of the cell structure. Examples of the polymer electrolyte include glycosaminoglycans such as heparin, chondroitin sulfate (for example, chondroitin 4-sulfate and chondroitin 6-sulfate), heparan sulfate, dermatan sulfate, keratan sulfate, and hyaluronic acid; and dextran sulfate, rhamnan sulfate, fucoidan, carrageenan, polystyrene sulfonic acid, polyacrylamide-2-methylpropanesulfonic acid, polyacrylic acid, and derivatives thereof, but the polymer electrolyte is not limited thereto. In the mixture prepared in the step (a), only one type of the polymer electrolyte may be mixed, or two or more types of the polymer electrolytes may be combined and mixed. In the construction of the cell structure according to the present embodiment, the polymer electrolyte is preferably glycosaminoglycan. In addition, it is more preferable to use at least one of heparin, dextran sulfate, chondroitin sulfate, or dermatan sulfate. It is still more preferable that the polymer electrolyte used in the present embodiment is heparin.

The amount of the polymer electrolyte mixed in the cationic buffer solution is not particularly limited as long as the growth of the cells and the construction of the cell structure are not adversely affected. For example, the concentration of the polymer electrolyte in the cationic buffer solution can be set to more than 0 mg/mL (higher than 0 mg/mL) and less than 1.0 mg/mL, and is preferably 0.025 to 0.1 mg/mL and more preferably 0.05 to 0.1 mg/mL. In addition, in the present embodiment, the cell structure can also be constructed by adjusting the mixture without mixing the polymer electrolyte.

As the extracellular matrix component used in the present embodiment, any component constituting an extracellular matrix (ECM) can be used as long as the component does not adversely affect the growth of cells and the formation of cell aggregates. Examples thereof include collagen, laminin, fibronectin, vitronectin, elastin, tenascin, entactin, fibrillin, proteoglycan, glycosaminoglycan, a modified form or variant thereof, and the like. Examples of the proteoglycan include a chondroitin sulfate proteoglycan, a heparan sulfate proteoglycan, a keratan sulfate proteoglycan, a dermatan sulfate proteoglycan, and the like. Examples of the glycosaminoglycan include hyaluronic acid, chondroitin 4-sulfate, chondroitin 6-sulfate, dermatan sulfate, keratan sulfate, heparan sulfate, heparin, and the like. In the mixture prepared in the step (a), only one type of the extracellular matrix component may be mixed, or two or more types of the extracellular matrix components may be combined and mixed. In the production of the cell structure according to the present invention, it is preferable to use one or more selected from the group consisting of collagen, laminin, and fibronectin, and among these, the collagen is preferable. As long as the growth of the cells and the formation of the cell structure are not adversely affected, a modified product or a variant of the above-described extracellular matrix component may be used.

The amount of the extracellular matrix component mixed in the cationic buffer solution is not particularly limited as long as the growth of the cells and the production of the cell structure are not adversely affected. For example, the concentration of the extracellular matrix component in the cationic buffer solution may be more than 0 mg/mL, and is preferably 0.010 mg/mL or more, more preferably 0.020 mg/mL or more, still more preferably 0.025 mg/mL or more, and even still more preferably 0.05 mg/mL or more. In addition, the concentration of the extracellular matrix component in the cationic buffer solution is preferably less than 1.0 mg/mL, more preferably 0.75 mg/mL or less, still more preferably 0.5 mg/mL or less, even still more preferably 0.25 mg/mL or less, and particularly preferably 0.1 mg/mL or less.

A blending ratio of the polymer electrolyte and the extracellular matrix component mixed in the cationic buffer solution is 1:2 to 2:1. In the production of the cell structure according to the present invention, a blending ratio of the polymer electrolyte and the extracellular matrix component is preferably 1:1.5 to 1.5:1, and more preferably 1:1.

A cell structure having a sufficient thickness can be constructed by repeating the steps (a) to (c), specifically, repeating seeding the mixture prepared in the step (a) on the cell structure obtained in the step (c) as the step (b) and then performing the step (c). A cell composition of a mixture newly seeded on the cell structure obtained in the step (c) may be the same as or different from the cell composition constituting the already constructed cell structure.

For example, first, a mixture containing only fibroblasts as the cells is prepared in the step (a), and the step (b) and the step (c) are performed to obtain a cell structure formed of 10 layers of fibroblast layers in the cell culture container. Next, a mixture containing only the vascular endothelial cells as the cells is prepared as the step (a), and the step (b) and the step (c) are performed to laminate one layer of the vascular endothelial cell layer on the fibroblast layer in the cell culture container. Furthermore, a mixture containing only fibroblasts as the cells is prepared as the step (a), and the step (b) and the step (c) are performed to laminate 10 layers of the fibroblast layers on the vascular endothelial cell layer in the cell culture container. Furthermore, a mixture containing cancer cells collected from a cancer patient is prepared as the step (a), and the step (b) and the step (c) are performed to laminate one layer of a cancer cell layer on the fibroblast layer in the cell culture container. As a result, a cell structure in which 10 layers of fibroblast layers, one layer of a vascular endothelial cell layer, 10 layers of fibroblast layers, and one layer of a cancer cell layer are sequentially laminated in a layer form for each cell type can be constructed. The thickness of the cell layer to be laminated in the step (c) can be adjusted by regulating the number of cells seeded in the step (b). The larger the number of cells seeded in the step (b) is, the larger the number of the cell layers laminated in the step (c) is. In addition, a mixture in which all the fibroblasts in 20 layers of the fibroblast layer and the vascular endothelial cells in one layer of the vascular endothelial cell layer are mixed is prepared in the step (a), the step (b) and the step (c) are performed, and the mixture containing the cancer cells collected from the cancer patient, which has been similarly prepared, is laminated on the formed multilayered structure, thereby a cell structure in which the cancer cell layer is laminated on the structure having a thickness of 21 layers and having a blood vessel network structure scattered inside the structure can be constructed. Furthermore, a mixture in which all the fibroblasts in 20 layers of the fibroblast layer, the vascular endothelial cells in one layer of the vascular endothelial cell layer, and the cells derived from cancer patient in one layer of the cancer cell layer are mixed is prepared in the step (a), and the step (b) and the step (c) are performed, thereby a cell structure having a thickness of 22 layers and in which both the cancer cells and the blood vessel network structure are independently scattered inside the structure can be constructed.

In a case where the steps (a) to (c) are repeated, the obtained cell structure may be cultured after the step (c) and before the step (b). A composition of a culture medium used for the culture, and culture conditions such as culture temperature, culture time, the atmosphere composition during the culture are set to conditions suitable for the culture of the cells constituting the cell structure. Examples of the culture medium include D-MEM, E-MEM, MEMα, RPMI-1640, Ham's F-12, and the like.

After the step (a), (a′-1) a step of removing a liquid portion from the obtained mixture to obtain a cell aggregate and (a′-2) a step of suspending the cell aggregate in a solution may be performed, and then the process may proceed to the step (b). The desired tissue body can be obtained by performing the above-described steps (a) to (c), but a more homogeneous tissue body can be obtained by performing (a′-1) and (a′-2) after the step (a) and performing the step (b).

As a means of removing the liquid portion in the step (a′-1), a method known to those skilled in the art can be used. For example, the liquid portion may be removed by centrifugation or filtration. Conditions for the centrifugation are not particularly limited as long as the growth of the cells and the formation of the cell aggregates are not adversely affected. For example, the liquid portion is removed by separating the liquid portion and the cell aggregate by subjecting the microtube containing the mixture to centrifugation at room temperature and 400×g for 1 minute. Alternatively, the liquid portion may be removed after the cells are collected by natural precipitation.

The solution used in the step (a′-2) is not particularly limited as long as the growth of the cells and the formation of the cell aggregates are not adversely affected. For example, a cell culture medium or a buffer solution suitable for the cells to be used is used.

In addition, after the step (a), the following steps (b′-1) and (b′-2) may be performed instead of the step (b). A more homogeneous tissue body can be obtained by performing the step (b′-1) and the step (b′-2). Also in the step (b′-2), precipitating the cell mixture seeded in the cell culture container in the cell culture container is included as in the step (b). The precipitation of the cell mixture may be actively performed by precipitating the cells by centrifugal separation or the like, or may be performed by natural precipitation. In the present embodiment and herein, the “cell viscous body” refers to a gel-like cell aggregate as described in Non-Patent Document 4.
(b′-1) A step of seeding a mixture obtained in the step (a) in a cell culture container, and then removing a liquid component from the mixture to obtain a cell viscous body, and
(b′-2) a step of suspending the cell viscous body in a solvent in the cell culture container.

The solvent for preparing the cell suspension is not particularly limited as long as the solvent is a solvent which is not toxic to the cells and does not impair the proliferative property or the function of the cells, and water, a buffer solution, a culture medium for cells, or the like can be used. Examples of the buffer solution include phosphate buffered saline (PBS), HEPES, Hank's buffer solution, and the like. Examples of the culture medium include D-MEM, E-MEM, MEMα, RPMI-1640, Ham's F-12, and the like. In a case where a culture medium for cells is used as a solvent for preparing the cell suspension, the cells can be cultured in a subsequent step (c) without removing the liquid components.

The following step (c′) may be performed instead of the step (c).
(c′): a step of forming a layer of cells on the substrate

In the step (c) and the step (c′), the liquid component may be removed from the seeded mixture. The method of the removal treatment of the liquid component in the step (c) and the step (c′) is not particularly limited as long as the growth of the cells and the construction of the cell structure are not adversely affected, and the removal treatment of the liquid component from the suspension of the liquid component and the solid component can be appropriately performed by a known method for those skilled in the art. Examples of the method include suction, centrifugal separation treatment, magnetic separation treatment, filtration treatment, and the like. For example, in a case where a cell culture insert is used as the cell culture container, the cell culture insert seeded with the mixture is subjected to a centrifugal separation treatment at 10° C. and 400×g for 1 minute to precipitate the cell mixture, thereby the liquid component can be removed by suction.

The culture medium used for the culture in the step (c) is not particularly limited as long as the culture medium is a medium in which the cells constituting the three-dimensional structure can grow, but a medium containing a low amount of growth factors such as an epithelial growth factor (EGF), VEGF, a fibroblast growth factor (FGF), and an insulin-like growth factor (IGF), or containing no growth factors, or a medium containing a low amount of serum or containing no serum is preferable. In a case where the content of various growth factors or the like is high, the vessel formation may be promoted. In the present embodiment, a medium to which a growth factor is not added is preferable, and a serum-free medium is more preferable.

### <Anticancer Drug>

In the evaluation method according to the present embodiment, the target anticancer drug to be evaluated for its drug efficacy need only be a drug used for cancer treatment, and is not limited to a drug that directly acts on cancer cells, such as a drug having cytotoxicity, and also includes a drug that does not have cytotoxicity but suppresses the proliferation of cancer cells, or the like.

Examples of the anticancer drug having no cytotoxicity include a drug that exhibits a function of suppressing the proliferation of cancer cells, slowing down the activity of cancer cells, or killing cancer cells by a synergistic action with immune cells in a living body or other drugs without directly attack on cancer cells, and a drug that suppresses the proliferation of cancer cells by impairing cells or tissues other than cancer cells. The anticancer drug used in the present embodiment may be a drug known to have an anticancer action, or may be a candidate compound for a novel anticancer drug.

The anticancer drug having cytotoxicity is not particularly limited, and examples thereof include molecular targeted drugs, alkylating agents, metabolic antagonists represented by a 5-FU-based anticancer drug, plant alkaloids, anticancer antibiotics, platinum derivatives, hormone agents, topoisomerase inhibitors, microtubule inhibitors, compounds classified as biological response modulators, and the like.

The anticancer drug that does not have cytotoxicity is not particularly limited, and examples thereof include an angiogenesis inhibitor, a prodrug of an anticancer drug, a drug that regulates the intracellular metabolic enzyme activity related to the metabolism of an anticancer drug or a prodrug thereof (hereinafter, referred to as “intracellular enzyme regulator” in the specification), and an immunotherapy drug (a drug that exerts an anticancer effect by improving the immune function, by activation of immune cells or motility of immune cells), and the like. In addition, examples thereof also include a drug that is finally involved in the anticancer action by enhancing the function of an anticancer drug or improving the immune function in a living body. The prodrug of the anticancer drug is a drug which is converted into an active form having an anticancer action by an intracellular enzyme of an organ such as a liver or a cancer cell. The cytokine network increases the enzyme activity of the intracellular enzyme, thus the amount of the active form is increased and the antitumor effect is enhanced, thereby examples of the drug involved in the anticancer action include the prodrug.

As the anticancer drug evaluated for drug efficacy in the evaluation method according to the present embodiment, the anticancer drug targeting a molecule constituting a signal transduction pathway stimulated by HGF, PIGF, VEGF, or bFGF, or a molecule activating the molecule is preferable because the effect of the present invention that an evaluation closer to the evaluation obtained in a case of using a cancer-bearing animal model is obtained is more sufficiently exhibited. Examples of the anticancer drug include a HER2 inhibitor, an ALK inhibitor, a BRAF inhibitor, a WNT inhibitor, and the like.

The anticancer drug evaluated for drug efficacy in the evaluation method according to the present embodiment may be one type, or two or more types of anticancer drugs may be used in combination. In addition, the anticancer drug may be used in combination with a drug other than the anticancer drug. For example, for the anticancer drug to be co-administered with the other drug in an actual clinical field, it is preferable that the evaluation method according to the present embodiment is carried out such that the anticancer drug and the other drug to be co-administered with the anticancer drug are used in combination, and the drug efficacy to be expected in the actual treatment can be evaluated with higher reliability.

### <Culture Step>

In the evaluation method according to the present embodiment, first, as a culture step, the cell structure according to the present embodiment is cultured in the presence of an anticancer drug which is a drug efficacy evaluation target. Specifically, the cell structure according to the present embodiment is cultured in a culture medium mixed with an anticancer drug. The anticancer drug may be added to a culture medium for culturing the cell structure at the same time as the start of the culture, or may be added at an appropriate time after the start of the culture. In addition, in a case of evaluating two or more types of drugs (namely, a combination of two or more types of anticancer drugs, or a combination of one or more types of anticancer drugs and one or more types of non-anticancer drugs), two or more types of drugs may be added to the culture medium of the cell structure at the same time, or each drug may be added independently.

The amount of the anticancer drug to be mixed with the culture medium can be experimentally determined in consideration of conditions such as the type and number of cells constituting the cell structure, the type and amount of cancer cells contained, the type of the culture medium, the culture temperature, and the culture time. For example, the culture time is not particularly limited, and can be set to 24 to 96 hours, and is preferably 48 to 96 hours and more preferably 48 to 72 hours. In addition, it is also possible to add, as necessary, hydrodynamic flow such as reflux as it does not significantly change the culture environment.

### <Evaluation Step>

An anticancer effect of an anticancer drug is evaluated using, as an index, the number of cells having a proliferation ability in cancer cells in the cell structure after the culture step. The anticancer effect means an effect of inhibiting proliferation of cancer cells or killing cancer cells.

Specifically, in a case where the number of cells having a proliferation ability in the cancer cell in the cell structure is small as compared with a case where the cell structure is cultured in an environment in which the anticancer drug is not present, the anticancer drug used is evaluated to have an anticancer effect on the cancer cell contained in the cell structure. On the other hand, in a case where the number of cells having a proliferation ability in the cancer cell is almost the same or large as compared with a case where the cell structure is cultured in an environment in which the anticancer drug is not present, the anticancer drug is evaluated to have no anticancer effect on the cancer cell contained in the cell structure.

The number of cells having a proliferation ability in the cancer cell can be evaluated using a signal correlated with the number of cancer cells having a proliferation ability or the abundance of the cancer cells having a proliferation ability. It is sufficient that the number of cancer cells having proliferation ability at the evaluation time point can be measured, and it is not always necessary to measure the number of cancer cells in a living state. For example, the cancer cell can be labeled to be distinguished from the other cells, and the signal from the label can be examined as an indicator. For example, after the cancer cell is fluorescent-labeled, the viability of the cells is determined and the number of cells determined to be viable cells can be directly counted as the cancer cells having proliferation ability in the cell structure. In this case, an image analysis technique can also be used. The determination of the viability of the cell can be performed by a known method for determining the viability of a cell, such as trypan blue staining or propidium iodide (PI) staining. The fluorescent labeling of the cancer cell can be performed by a known method such as an immunostaining method using, for example, an antibody against a substance specifically expressed on the cell surface of the cancer cell as a primary antibody and a fluorescent-labeled secondary antibody that specifically binds to the primary antibody. The determination of the viability of the cell and the measurement of the number of viable cells may be performed in a state of the cell structure, or may be performed in a state in which the cell structure is destroyed at a single cell level. For example, after destroying the three-dimensional structure of the cell structure after labeling cancer cells and dead cells, only the cancer cells that has been alive at the evaluation time point can be directly counted by fluorescence activated cell sorting (FACS) or the like using the labeling as an index.

By labeling the cancer cell in the cell structure in a living state and detecting a signal from the labeling over time, the number of cells having proliferation ability of the cancer cell in the cell structure can also be measured over time. The cancer cell in the cell structure may be labeled after the cell structure is constructed, or the cancer cell may be labeled in advance before the cell structure is constructed. For example, in a case where a cell structure including a cell group including cancer cells derived from a cancer patient is used, the cancer cells can be labeled in advance before the cell structure is constructed. In addition, other cells derived from a cancer patient may be labeled in the same manner together with the cancer cells. Furthermore, in a case where cancer cells that constantly express the fluorescent dye are used, the number of viable cancer cells can be evaluated by measuring the fluorescence intensity of the lysate obtained by lysing the cell structure with a microplate reader or the like.

In the evaluation method according to the present embodiment, a cell structure containing stromal cells that secrete a growth factor such as HGF, similar to the structure of the peripheral tissue of the cancer cell in an actual living body is used and evaluation is performed in a state in which an environment closer to in vivo is simulated in vitro, thereby evaluation for drug efficacy with high reliability can be obtained. In a case where the anticancer drug that has been evaluated to have an anticancer effect by the evaluation method according to the present embodiment is actually administered to a cancer patient, a sufficient anticancer effect can be expected to be obtained. Therefore, the evaluation method according to the present embodiment can be used as an in vitro drug evaluation tool that has not been used in the past in screening of an anticancer drug candidate compound in a drug discovery field, drug repositioning screening, selection and determination of an anticancer drug treatment method (single-treatment/combination treatment) in a clinical field (anticancer drug sensitivity test), and the like. In particular, the anticancer drug that has been evaluated to have an anticancer effect by performing the evaluation method according to the present embodiment using a cell structure containing cancer cells collected from a cancer patient can be expected to exhibit an appropriate anticancer effect in a case of being actually administered to the cancer patient.

As another aspect, a selection method of an anticancer drug according to a second embodiment includes culturing a cell structure containing a cancer cell and a stromal cell in a presence of one or two or more of anticancer drugs and selecting an anticancer drug as an anticancer drug having an anticancer effect to the cancer cells when, using the number of cells having a proliferation ability of the cancer cells in the cell structure after the culturing as an index, the number of cells having a proliferation ability of the cancer cells in the cell structure is less than the case where the cell structure is cultured under the condition where the anticancer drug does not exist, in which a signal transduction pathway in the cancer cells, stimulated by a hepatocyte growth factor, a placental growth factor, a vascular endothelial growth factor, or a basic fibroblast growth factor is more activated than a signal transduction pathway in a normal cell.

The second embodiment differs from the first embodiment in that the second embodiment includes a step of selecting the anticancer drug rather than the step of evaluating in the first embodiment. Other than this, the first and second embodiments are the same to one another, therefore, the explanations indicated in the first embodiment are incorporated by reference and the explanations are omitted.

The selection method of the anticancer drag according to the second embodiment includes selecting the anticancer drug as an anticancer drug having the anticancer effect to the cancer cells when, using the number of cells having a proliferation ability of the cancer cells in the cell structure after the culturing as an index, the number of cells having a proliferation ability of the cancer cells in the cell structure is less than the case where the cell structure is cultured under the condition where the anticancer drug does not exist, in which a signal transduction pathway in the cancer cells.

On the other hand, comparing to the case where the cell structure is cultured under the condition where the anticancer drug, the number of cells having a proliferation ability of the cancer cells in the cell structure is almost the same or large, the anticancer drug does not have the anticancer effect to the cancer cells included in the cell structure and is not selected as an anticancer drug.

The number of cells having a proliferation ability of the cancer cells can be measured by the method described on the first embodiment.

In the selection method according to the present embodiment, a cell structure containing stromal cells that secrete a growth factor such as HGF, similar to the structure of the peripheral tissue of the cancer cell in an actual living body is used, and the anticancer drug with drug efficacy with high reliability can be selected because the effect of the anticancer drug is confirmed in a state in which an environment closer to in vivo is simulated in vitro. In a case where the anticancer drug that has been selected to have an anticancer effect by the selection method according to the present embodiment is actually administered to a cancer patient, a sufficient anticancer effect can be expected to be obtained. Therefore, the selection method according to the present embodiment can be used as an in vitro drug evaluation tool that has not been used in the past in screening of an anticancer drug candidate compound in a drug discovery field, drug repositioning screening, selection and determination of an anticancer drug treatment method (single-treatment/combination treatment) in a clinical field (anticancer drug sensitivity test), and the like. In particular, the anticancer drug that has been selected to have an anticancer effect by performing the selection method according to the present embodiment using a cell structure containing cancer cells collected from a cancer patient can be expected to exhibit an appropriate anticancer effect in a case of being actually administered to the cancer patient.

### EXAMPLE

Hereinafter, the present invention will be described in more detail with reference to Examples, but the present invention is not limited to Examples.

### <Reagent and the Like>

In the following experiments, the following reagents and the like were used.

TABLE 1

| Reagent name | Manufacturer | Product number |
|---|---|---|
| D-MEM (high glucose) | FUJIFILM Wako Pure | 043-30085 |
| | Chemical Corporation | |
| EGM2 | Lonza | CC3202 |
| FBS | Gibco | 10437028 |
| 100X Penicillin/Streptomycin solution | FUJIFILM Wako Pure | 168-23191 |
| | Chemical Corporation | |
| D-PBS (−) | FUJIFILM Wako Pure | 045-29795 |
| | Chemical Corporation | |
| RPMI1640 | FUJIFILM Wako Pure | 189-02025 |
| | Chemical Corporation | |
| Ham's F12 | FUJIFILM Wako Pure | 087-08335 |
| | Chemical Corporation | |
| HEPES buffer solution | FUJIFILM Wako Pure | 345-06681 |
| | Chemical Corporation | |
| Penicillin-Streptomycin-Amphotericin | FUJIFILM Wako Pure | 161-23181 |
| | Chemical Corporation | |
| 0.25% Trypsin-EDTA solution | Invitrogen | 25200072 |
| 1x TrypLETM Express | Invitrogen | 12604013 |
| Collagen I | Nippi, Incorporated | ASC-1-100-100 |
| Heparin | sigma-Aldrich | H3393-100KU |
| Ultra Pure 1M Tris-HCl pH 7.5 | Invitrogen | 15567-027 |
| 1 mol/L Acetic acid | FUJIFILM Wako Pure | 013-18845 |
| | Chemical Corporation | |
| Sterilized water | FUJIFILM Wako Pure | 039-24155 |
| | Chemical Corporation | |
| Fibronectin | sigma-Aldrich | F1141-5MG |
| HBSS-Hank's balanced salt solution | Gibco | 14170-120 |
| Matrigel | Corning Incorporated | 356237 |
| Mildform (R) 10N_1L_for pathology research | FUJIFILM Wako Pure | 133-10311 |
| | Chemical Corporation | |
| Ethanol (99.5) | FUJIFILM Wako Pure | 052-07221 |
| | Chemical Corporation | |
| Triton X-100 | FUJIFILM Wako Pure | 160-24751 |
| | Chemical Corporation | |
| 30 w/v % Albumin D-PBS(−) solution, from | FUJIFILM Wako Pure | 015-23871 |
| bovine serum (BSA), fatty acid free | Chemical Corporation | |
| Visikol Histo #1 | Visikol Inc. | VSK-H1-100-100 |
| Anti-cytokeratin (CK) 8/18 antibody, rabbit | Dako | M365201 (Clone |
| monoclonal | | EP17/EP30) |
| Anti-human Ki-67 antibody, mouse | Dako | M724001-2 (Clone |
| monoclonal | | MIB-1) |
| Anti-mouse IgG (H + L) antibody, Alexa Flour ® | Invitrogen | A11005 |
| 594, goat monoclonal | | |
| Anti-rabbit IgG (H + L) antibody, Alexa Flour ® | Invitrogen | A21244 |
| 647, goat monoclonal | | |

TABLE 2

| Reagent name | Manufacturer | Product number |
|---|---|---|
| Tumor & Tissue DissociationReagent | BD Biosciences | 661563 |
| Tumor Cell Isolation Kit, human | Miltenyi | 130-108-339 |
| LS Columns | Miltenyi | 130-042-401 |
| Lysis Buffer | Laboratory-developed reagent | |
| BCA assay kit | FUJIFILM Wako Pure Chemical | 297-73101 |
| | Corporation | |
| Albumin solution | FUJIFILM Wako Pure Chemical | 015-25613 |
| | Corporation | |
| Sample buffer | nacalai tesque | 949914 |
| Molecular weight marker | Cytiva | RPN800E |
| Gel for SDS-PAGE | FUJIFILM Wako Pure Chemical | 194-15021 |
| | Corporation | |
| 10X TBS Buffer | NIPPON GENE CO., LTD. | 317-90175 |
| SDS-PAGE Running Buffer | NIPPON GENE CO., LTD. | 318-90323 |
| Transfer buffer | FUJIFILM Wako Pure Chemical | 015-26213 |
| | Corporation | |
| Can get signal | TOYOBO CO., LTD. | NKB-101 |
| BSA | sigma-Aldrich | 015-23871 |
| Stripping solution | FUJIFILM Wako Pure Chemical | 193-16375 |
| | Corporation | |
| Polyoxyethylene (20) sorbitan monolaurate | FUJIFILM Wako Pure Chemical | 160-21211 |
| | Corporation | |
| anti-GAPDH mouse antimbody | FUJIFILM Wako Pure Chemical | 016-25523 |
| | Corporation | |
| anti-Her2 rabbit antibody | Cell signaling technology | 2165S |
| anti-pHer2 rabbit antibody | Cell signaling technology | 2243S |
| anti-Akt rabbit antibody | Cell signaling technology | 4691S |
| anti-pAkt rabbit antibody | Cell signaling technology | 4060S |
| anti-ERK rabbit antibody | Cell signaling technology | 9102S |
| anti-pERK rabbit antibody | Cell signaling technology | 9101S |
| anti-S6 rabbit antibody | Cell signaling technology | 2217S |
| anti-pS6 rabbit antibody | Cell signaling technology | 5364S |
| Anti-Rabbit IgG, HRP-Linked Whole Ab | GE HealthCare Technologies Inc. | NA934-1ML |
| Donkey | | |
| Anti-Mouse IgG, HRP-Linked Whole Ab | GE HealthCare Technologies Inc. | NA931-1ML |
| Sheep | | |
| Super signal | Thermo Fisher Scientific | 34095 |

TABLE 3

| Reagent name | Manufacturer | Product number |
|---|---|---|
| Growth Factor array | Abcam | ab134002 |
| HGF, Human, ELISA Kit | R&D Systems | DHG00B-1 |
| VEGF, Human, ELISA Kit | R&D Systems | DVE00-1 |
| FGFb, Human, ELISA Kit | R&D Systems | DFB50 |
| PIGF, Human, ELISA Kit | R&D Systems | DPG00-1 |

### Example 1

The drug efficacy evaluation of the anticancer drug on the cancer cell was performed by using a two-dimensionally cultured cancer cell, by using a three-dimensionally cultured cell structure, and by using a cancer-bearing model mouse, and was compared.

### <Evaluation of Drug Sensitivity (3D Culture)>

Using human umbilical vein endothelial cells (HUVEC) (manufactured by Lonza Group AG) as a vascular endothelial cell, human newborn dermal fibroblasts (NHDF) (manufactured by Lonza Group AG) as an stromal cell, and two strains of human non-small cell lung cancer cells having HER2 mutation (LCC007 strain and LCC381 strain) as a cancer cell, a cell structure in which a layer of cancer cells was laminated on the top surface of a layer having a thickness of 5 m or more consisting of HUVEC and NHDF was constructed, and the anticancer effect of Lapatinib, which is a HER2 inhibitor, was evaluated.

### (Construction of Cell Structure)

HUVEC was mixed with NHDF such that HUVEC was 1.5% (number of cells/number of cells) with respect to NHDF, and centrifuged, and the supernatant was removed, and then Hep/Col solution (a mixture solution of heparin and collagen) was added thereto. Next, the supernatant was removed after centrifugation, and then the cells were suspended in a culture medium. The suspension was performed such that the total liquid amount was 135 μL per 0.9×10⁶ particles. The cells suspended in the culture medium were seeded in a transwell insert-integrated 96-well (0.4 μm polyester membrane) treated with fibronectin coding, and the cells were precipitated by plate centrifugation (400 G, 2 minutes) and cultured in an incubator at 37° C. in an atmosphere of 5% CO₂ to obtain a cell structure consisting of NHDF and HUVEC. On the next day of the start date of the culture, an appropriate amount of cancer cells was seeded on the top surface of the cell structure and cultured to construct a cell structure containing cancer cells, NHDF, and HUVEC. At a time point of fifth day from the day of seeding of NHDF and HUVEC (the day of constructing the cell structure), a drug treatment was performed by adding a drug (anticancer drug) solution to the culture medium. After culturing for 3 days from the addition of the anticancer drug (eighth day from the day of constructing the cell structure), the cell structure was fixed with 10% formalin treatment. A cell structure cultured in the same manner as described above, except that only the solvent of the solution was added instead of the anticancer drug solution, was used as a control.

### (Fluorescent Immunostaining)

The cell structure after formalin fixation was immersed in PBS(−) containing 1% BSA and 0.2% Triton X at room temperature for 1 hour to perform membrane permeabilization treatment and blocking. Next, the primary antibody diluted to a designated concentration with PBS(−) containing 1% BSA and 0.2% Triton X, and DAPI were added thereto, and the cell structure was allowed to stand at 4° C. overnight to perform a primary antibody reaction. Subsequently, the cell structure was washed three times with PBS(−), and then a secondary antibody diluted to a designated concentration with PBS(−) containing 1% BSA and 0.2% Triton X was added thereto, and the cell structure was allowed to stand at room temperature for 1 hour with light shielding to perform a secondary antibody reaction. Thereafter, the cell structure was washed with PBS (−), and immersed in 99.5% ethanol to perform a dehydration treatment and a transparent treatment.

### (Fluorescent Image Acquisition and Analysis)

A fluorescence-stained image of the sample after the immunostaining was acquired by a confocal imaging system (Operetta CLS, manufactured by PerkinElmer, Inc.). The acquired image was analyzed by image analysis software (Harmony). A range in which the cancer cells were present was set as a first region of interest (ROI) in a circular shape and the CK8/18 expression region was recognized. The recognition site of CK8/18 was set as the second ROI and the Ki-67 expression region was recognized. The staining area Confluency of CK8/18 and the recognition Object number of Ki-67 for the first ROI were calculated. The number of cancer cells in the control in which the anticancer drug was not added was set to 100%, and the relative number (%) of the surviving cancer cells after treatment with the anticancer drug at each concentration for 3 days was defined as the relative cancer cell survival rate (%).

### <Evaluation of Drug Sensitivity (Spheroid Culture)>

The evaluation of the drug sensitivity of the spheroid-cultured cells was performed by an ATP assay. First, the cancer cells were seeded on a super low attachment surface round bottom black plate (transparent bottom) at an appropriate seeding number determined in advance. The next day or 3 days after the seeding, it was confirmed that the cancer cells aggregated to form a spherical cell mass (spheroid) using a phase-contrast microscope. Thereafter, an anticancer drug solution (a solution containing an anticancer drug in ⅓ amount of the culture medium at the time of seeding) was added to the culture medium to perform drug treatment. After culturing for 3 days from the addition of the anticancer drug, a kit for a bioluminescence assay “CellTiter-Glo (registered trademark)” was added to the culture medium, and the chemiluminescence was detected after shaking at room temperature for 10 minutes.

### <Evaluation of Drug Sensitivity (2D Culture)>

The evaluation of drug sensitivity by the cancer cells that had been two-dimensionally cultured (2D culture) was performed by an ATP assay. First, the cancer cells were seeded on a black plate (transparent bottom) at an appropriate seeding number determined in advance. On the next day of the seeding, an anticancer drug solution (a solution containing an anticancer drug in ⅓ amount of the culture medium at the time of seeding) was added to the culture medium to perform drug treatment. A 2D-cultured cells in the same manner as described above, except that only the solvent of the solution was added instead of the anticancer drug solution, was used as a control. After culturing for 3 days from the addition of the anticancer drug, a kit for a bioluminescence assay “CellTiter-Glo (registered trademark)” was added to the culture medium, and the chemiluminescence was detected after shaking at room temperature for 10 minutes. The amount of chemiluminescence of the cells in the control in which the anticancer drug was not added was set to 100%, and the relative amount of chemiluminescence (%) of the cells after treatment with the anticancer drug at each concentration for 3 days was defined as the relative cancer cell survival rate (%).

### <Evaluation of Drug Sensitivity (2D Culture in Case of Adding 3D Cell Tissue Supernatant)>

The drug sensitivity in a case where the culture supernatant in a case of the 3D culture was added to the culture medium of the 2D culture and the culture was performed was evaluated. Specifically, evaluation of drug sensitivity (3D culture) was performed in the same manner as the <Evaluation of drug sensitivity (2D culture)> described above, except that, as the anticancer drug solution, a solution obtained by adding an anticancer drug to the culture supernatant in the case of the 3D culture in an amount corresponding to ⅓ of the amount at the time of seeding was used.

### <Evaluation of Drug Sensitivity (In Vivo)>

The anticancer drug was administered to the cancer-bearing mouse produced by subcutaneously transplanting the cancer cells (LCC007 strain or LCC381 strain) into a nude mouse, and the drug sensitivity was evaluated.

### (Production of Cancer-Bearing Mouse)

A cancer-bearing mouse was produced by subcutaneously transplanting the cancer cells to a nude mouse (phyletic line: BALB/c-nu (nu/nu), sex: female, age: 5 weeks) purchased from The Jackson Laboratory Japan at 10×10⁶ cells/mouse. The cancer cells were cultured in an amount that could be transplanted in advance, recovered, then suspended in 50 μL/mouse of Matrigel, and administered to a mouse.

### (Measurement of Body Weight and Tumor Diameter, and Grouping)

Prior to the administration of the anticancer drug, a short diameter and a long diameter of the subcutaneous tumor were measured, and a tumor volume was calculated from the following expression. In addition, the tumor diameter and the body weight were measured at a frequency of twice a week or more after start date (day 0) of the grouping and the administration. The grouping was performed at a time point at which the average tumor volume was about 50 to 100 mm³. The grouping was performed such that the average tumor volume of each group was approximated, and 6 mice were allocated to each of the vehicle group and the drug treatment group.

$$[ Tumor volume ( {mm}^{3} ) ] = 1 / 2 \times L \times W \times W$$

L: tumor long diameter (mm), W: tumor short diameter (mm)

### (Preparation and Administration of Drug)

The drug (Lapatinib) was orally administered to a cancer-bearing mouse, and the tumor volume and the body weight were measured over time. Table 4 shows the solvent, dose, and administration cycle of the drug administered to the cancer-bearing mouse transplanted with the LCC07 strain. Table 5 shows the solvent, dose, and administration cycle of the drug administered to the cancer-bearing mouse transplanted with the LCC381 strain. The administration amount was calculated from the body weight for each individual as 10 μL/g (body weight) and administered. On the fourteenth day from the start of the administration, the mouse was euthanized by central disruption (cervical dislocation).

TABLE 4

| Drug | | | Administration |
|---|---|---|---|
| name | Solvent | Dose | cycle |
| Vehicle | 20% PEG400 + 3% Tween 80 | — | 5/week, 1/day |
| Lapatinib | 20% PEG400 + 3% Tween 80 | 30 mg/kg | 5/week, 1/day |

TABLE 5

| Drug | | | Administration |
|---|---|---|---|
| name | Solvent | Dose | cycle |
| Vehicle | Solution of | — | 5/week, 1/day |
| | DMSO:20% SBE-β-CD = 1:9 | | |
| Lapatinib | 20% PEG400 + 3% Tween 80 | 30 mg/kg | 5/week, 1/day |

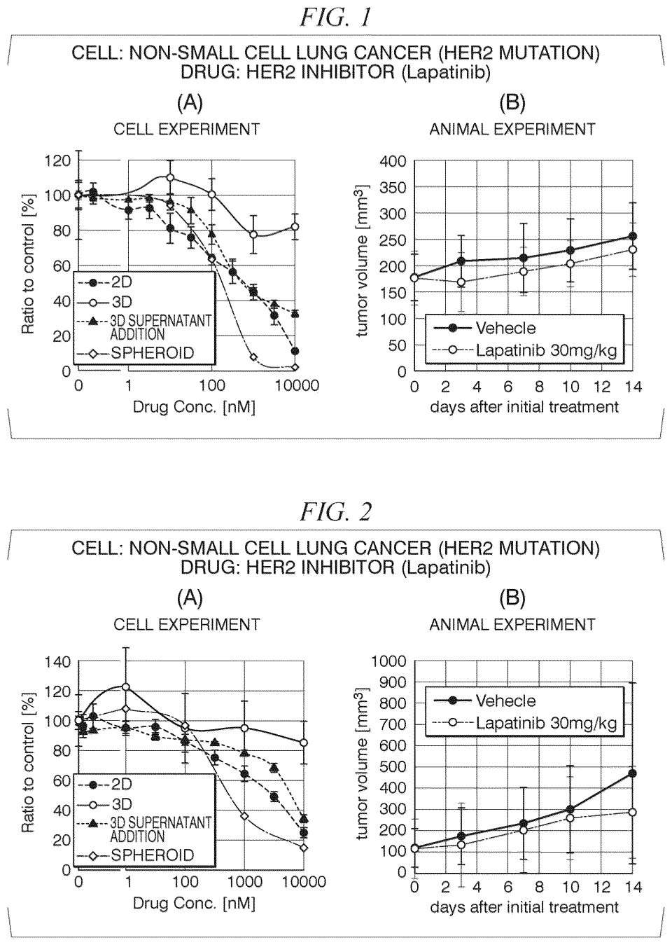

FIG. 1 shows the results of sensitivity evaluation of the LCC007 strain to Lapatinib, and FIG. 2 shows the results of sensitivity evaluation of the LCC381 strain to Lapatinib. (A) of FIG. 1 and (A) of FIG. 2 show evaluation results of the cell experiment (in vitro), that is, results of measuring the relative cell survival rate (%) of each cell group over time, and (B) of FIG. 1 and (B) of FIG. 2 show evaluation results of the animal experiment (in vivo), that is, results of measuring the tumor volume of each mouse group over time. As shown in (A) of FIG. 1 and (A) of FIG. 2, in vitro, in both cancer cell strains, the relative cell survival rate decreased in a concentration-dependent manner of the added anticancer drug in the spheroid culture and the 2D culture, but the survival rate did not decrease in the 3D culture even in a case where the concentration of the anticancer drug was increased. In the 2D culture in a case of adding 3D cell tissue supernatant (in the figure, “3D supernatant addition”), the cell survival rate was slightly higher than that in the 2D culture. On the other hand, as shown in (B) of FIG. 1 and (B) of FIG. 2, in vivo, although the tumor volume of the Lapatinib administration group was slightly smaller than that of the Vehicle group, a significant anticancer effect was not confirmed. From these results, it was confirmed that, in the 2D culture or the spheroid culture, the same evaluation as the evaluation obtained in a case of using a cancer-bearing animal model may not be obtained, and by using the cell structure according to the present embodiment, an evaluation closer to the evaluation obtained in a case of using a cancer-bearing animal model can be obtained.

### Example 2

A growth factor in the culture supernatant of the cell structure containing the stroma was examined.

### (Construction of Cell Structure and Recovery of Culture Supernatant)

HUVEC was mixed with NHDF such that HUVEC was 1.5% (number of cells/number of cells) with respect to NHDF, and centrifuged, and the supernatant was removed, and then Hep/Col solution (a mixture solution of heparin and collagen) was added thereto. Next, the supernatant was removed after centrifugation, and then the cells were suspended in a culture medium. The suspension was performed such that the total liquid amount was 135 μL per 0.9×1⁰⁶ particles. The cells suspended in the culture medium were seeded in a transwell insert-integrated 96-well (0.4 μm polyester membrane) treated with fibronectin coding, and the cells were precipitated by plate centrifugation (400 G, 2 minutes) and cultured in an incubator at 37° C. in an atmosphere of 5% CO₂ to obtain a cell structure consisting of NHDF and HUVEC. On the next day of the start date of the culture, an appropriate amount of cancer cells was seeded on the top surface of the cell structure and cultured to construct a cell structure containing cancer cells, NHDF, and HUVEC. Two days or five days after the day (the day of constructing the cell structure) of seeding NHDF and HUVEC, the culture medium was exchanged with D-MEM not containing FBS. In a case of exchanging the culture medium, the culture medium was exchanged after washing the cells twice with PBS. On the third day from the medium exchange, the culture supernatant was recovered, and the solid content such as cells was removed through a filter to obtain a sample.

### (2D Culture and Recovery of Culture Supernatant)

NHDF and HUVEC were seeded on a 90φ dish at the same ratio as the 3D cell tissue. Two days after the seeding, the culture medium was exchanged with D-MEM not containing FBS. In a case of exchanging the culture medium, the culture medium was exchanged after washing the cells twice with PBS. On the third day from the medium exchange, the culture supernatant was recovered, and the solid content such as cells was removed through a filter to obtain a sample.

### (Analysis of Culture Supernatant)

As a control sample, D-MEM not containing FBS was used, the sample acquired above was supplied to a growth factor analysis array kit “Growth factor array” to detect the contained protein, and the amount of the contained protein was measured using various ELISA kits. In the growth factor array and various ELISAs, the work was performed according to the attached procedure manual of the kit.

As a result of the analysis of the “growth factor array”, the 2D culture supernatant did not contain any growth factors, whereas the 3D culture supernatant contained HGF and PIGF in both the fifth day of culture and the eighth day of culture, and also contained VEGF in the culture supernatant of the fifth day of culture. In addition, the amount of the growth factor in the 3D culture supernatant was not different between the cell structure consisting of NHDF and HUVEC and the cell structure consisting of NHDF, HUVEC, and the cancer cells (LCC007 strain or LCC0381 strain).

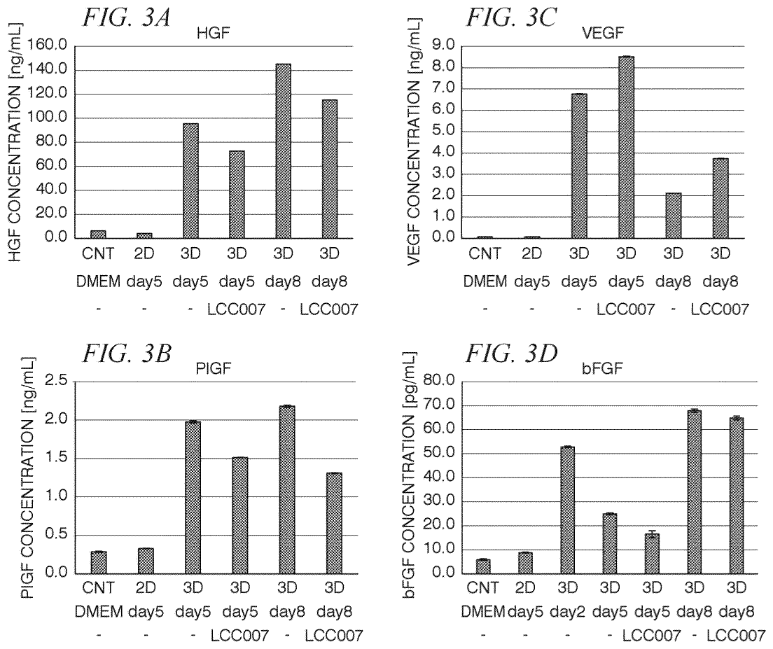

In addition, the amounts of HGF, VEGF, PIGF, and bFGF in each culture supernatant were measured. The results are shown in FIG. 3. In FIG. 3, “CNT” represents the result of the culture medium (D-MEM), “2D” represents the result of the 2D culture supernatant, and “3D” represents the result of the 3D culture supernatant. In the figure, “day 5” represents the result of the culture supernatant on the fifth day of culture, and “day 8” represents the result of the culture supernatant on the eighth day of culture. As a result, all of the growth factors were hardly contained in the 2D culture supernatant, but were contained in the 3D culture supernatant. In addition, the concentration of HGF ((A) of FIG. 3) and the concentration of PIGF ((C) of FIG. 3) were decreased in the culture supernatant of the cell structure containing the cancer cells as compared with the culture supernatant of the cell structure not containing the cancer cells, but the concentration of VEGF ((B) of FIG. 3) and the concentration of bFGF ((D) of FIG. 3) were increased in the culture supernatant of the cell structure containing the cancer cells as compared with the culture supernatant of the cell structure not containing the cancer cells. As described above, in a case of co-culturing with the cancer cells, a tendency of decrease or increase in the concentration of each growth factor was observed.

### Example 3

A state of activation of an intracellular signal transduction pathway stimulated by HGF in the cells of the cell structure containing the stroma was examined. As the cancer cell, a non-small cell lung cancer cell LCC007 strain was used.

### (Construction, Culture, and Sample Preparation of Cell Structure)

HUVEC was mixed with NHDF such that HUVEC was 1.5% (number of cells/number of cells) with respect to NHDF, and centrifuged, and the supernatant was removed, and then Hep/Col solution (a mixture solution of heparin and collagen) was added thereto. Next, the supernatant was removed after centrifugation, and then the cells were suspended in a culture medium. The suspension was performed such that the total liquid amount was 135 μL per 0.9×10⁶ particles. The cells suspended in the culture medium were seeded in a transwell insert-integrated 96-well (0.4 μm polyester membrane) treated with fibronectin coding, and the cells were precipitated by plate centrifugation (400 G, 2 minutes) and cultured in an incubator at 37° C. in an atmosphere of 5% CO₂ to obtain a cell structure consisting of NHDF and HUVEC. On the next day of the start of the culture, an appropriate amount of cancer cells (LCC007 strain) was seeded on the top surface of the cell structure and cultured, thereby a cell structure containing the cancer cells, NHDF, and HUVEC was constructed. Treatment with a drug (Lapatinib) was performed 7 days or 8 days after the day of seeding NHDF and HUVEC (the day of constructing the cell structure), and the cells treated with the drug on the seventh day from the construction of the cell structure were trypsinized 24 hours after the treatment, and the cells treated with the drug on the eighth day from the construction of the cell structure were trypsinized 6 hours after the treatment, and the cell structures were peeled off from the polyester membrane and recovered. The recovered cell structure was treated with a dephosphorylation inhibitor, and then cancer cells were concentrated using a Tumor isolation kit. Lysis Buffer was added to the concentrated cancer cells, and the mixture was heated at 100° C. for 3 minutes and then subjected to ultrasonic treatment until the liquid viscosity was further eliminated. Next, the supernatant was recovered by performing a centrifugal separation treatment (13,500 rpm, 10 minutes). The protein mass contained in the recovered supernatant was quantified, and the supernatant was diluted with a sample buffer such that the protein amount per lane was constant (2 to 4 μg/lane), then allowed to stand at 100° C. for 3 minutes to perform a reduction treatment, and used as a sample.

### (Sample Preparation of 2D-Cultured Cells)

The cancer cells were seeded on a 90φ dish. Treatment with a drug was performed 7 days or 8 days after seeding, and the cells treated with the drug 7 days after seeding were trypsinized 24 hours after the treatment, and the cells treated with the drug 8 days after seeding were trypsinized 6 hours after the treatment, and the cells were peeled off and recovered. Lysis Buffer was added to the recovered cells, and the mixture was heated at 100° C. for 3 minutes and then subjected to ultrasonic treatment until the liquid viscosity was further eliminated. Next, the supernatant was recovered by performing a centrifugal separation treatment (13,500 rpm, 10 minutes). The protein mass contained in the recovered supernatant was quantified, and the supernatant was diluted with a sample buffer such that the protein amount per lane was constant (2 to 4 μg/lane), then allowed to stand at 100° C. for 3 minutes to perform a reduction treatment, and used as a sample.

### (Sample Preparation of Cells of Cancer-Bearing Mouse (In Vivo))

The mouse after the end of the test in <Evaluation of drug sensitivity (in vivo)> of Example 1 was euthanized by central disruption (cervical dislocation). Thereafter, the subcutaneous tumor was excised and stored at −80° C. The stored tumor was dissolved in an appropriate amount of Lysis Buffer, heated at 100° C. for 3 minutes, and then further subjected to ultrasonic treatment until the liquid viscosity was eliminated. Next, the supernatant was recovered by performing a centrifugal separation treatment (13,500 rpm, 10 minutes). The protein mass contained in the recovered supernatant was quantified, and the supernatant was diluted with a sample buffer such that the protein amount per lane was constant (2 to 4 μg/lane), then allowed to stand at 100° C. for 3 minutes to perform a reduction treatment, and used as a sample.

### (Western Blotting)

The prepared sample and the molecular weight marker were loaded onto a gel for SDS-PAGE, and electrophoresis was performed. The protein was transferred from the gel after electrophoresis to a PVDF membrane. The transferred membrane was immersed in 4% BSA/PBS, blocked by shaking at room temperature for 1 hour, and then subjected to a primary antibody reaction. The primary antibody reaction was performed by shaking at room temperature for 1 hour or by allowing to stand at 4° C. overnight. After washing the membrane, the secondary antibody reaction was performed by shaking at room temperature for 1 hour. After washing the membrane, the membrane was allowed to emit light using a detection reagent and was imaged with an imager (AI600, manufactured by Cytiva). The captured image was appropriately subjected to image processing using the image analysis software Photoshop.

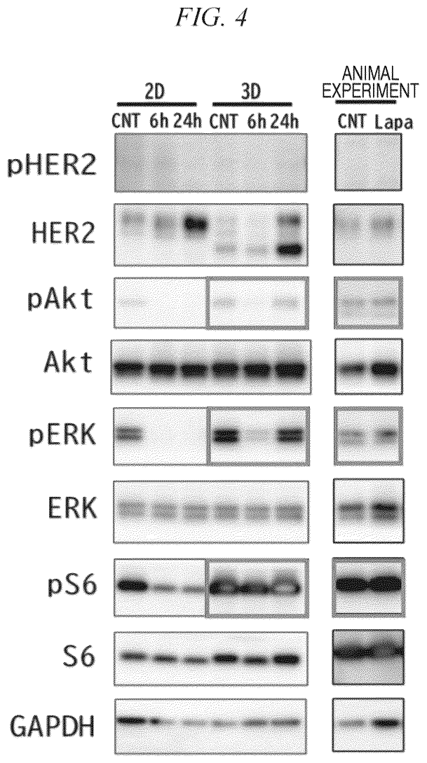

FIG. 4 shows an image of the membrane detected in the western blotting. As a result, in the sample derived from the cancer-bearing mouse, in both the Lapatinib-treated sample derived from the mouse (in the figure, lane of “Lapa” in “animal experiment”) and the untreated sample derived from the mouse (in the figure, lane of “CNT” in “animal experiment”), phosphorylated Akt (pAkt), phosphorylated ERK (pERK), and phosphorylated S6 (pS6) were detected. In the sample derived from the cells 2D-cultured, in the untreated sample (in the figure, lane of “2D” of “CNT”), bands of pAkt, pERK, and pS6 were detected, but in the cells treated with Lapatinib (in the figure, lanes of “6h” and “24h” of “2D”), bands of pAkt and pERK were not detected, and the band of pS6 was much thinner than that of the untreated sample. On the other hand, in the sample derived from the cell structure 3D-cultured, as in the sample derived from the cancer-bearing mouse, bands of pAkt, pERK, and pS6 were detected not only in the untreated sample (in the figure, lane of “CNT” of “3D”) but also in the sample treated with Lapatinib and cultured for 24 hours (in the figure, lane of “24h” of “3D”). In a sample derived from the cell structure 3D-cultured, which was treated with Lapatinib and cultured for 6 hours, a band of pS6 was detected, but bands of pAkt and pERK were not detected. From these results, it was confirmed that in the cell structure 3D-cultured, a growth factor such as HGF was secreted from the stromal tissue as in the body of the cancer-bearing mouse, and thus a downstream signal transduction pathway was activated, and on the other hand, in the 2D culture, the growth factor was not secreted, and thus the downstream signal transduction pathway was not activated.

### Example 4

The drug efficacy evaluation of the anticancer drug on the cancer cell was performed by using a two-dimensionally cultured cancer cell and by using a three-dimensionally cultured cell structure, and was compared.

As the cancer cells, an EML4-ALK positive non-small cell lung cancer cell LCC028-3 strain, a BRAF positive colon cancer cell JC215 strain, and a CD74-ROS1 positive non-small cell lung cancer cell LCC168 strain were used. As the anticancer drug, an ALK inhibitor (Alectinib), a BRAF inhibitor (Davrafenib), and a WNT inhibitor (FH535) were used. The evaluation of the sensitivity by the 2D culture and the 3D culture was performed in the same manner as in Example 1.

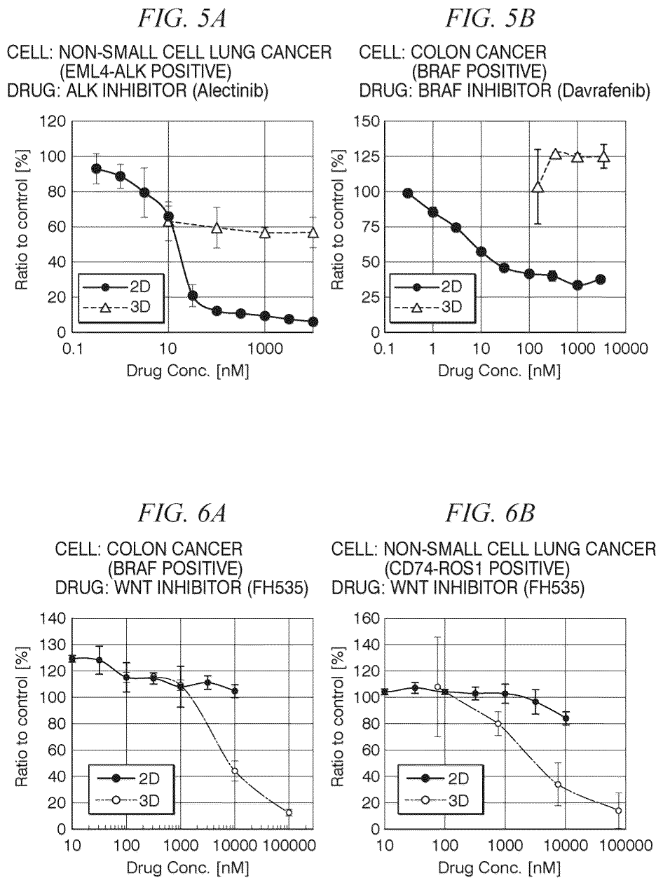

The sensitivity evaluation result of the EML4-ALK-positive non-small cell lung cancer cell LCC028-3 strain to the ALK inhibitor was shown in (A) of FIG. 5, the sensitivity evaluation result of the BRAF-positive colon cancer cell JC215 strain to the BRAF inhibitor was shown in (B) of FIG. 5, the sensitivity evaluation result of the BRAF-positive colon cancer cell JC215 strain to the WNT inhibitor was shown in (A) of FIG. 6, and the sensitivity evaluation result of the CD74-ROS1-positive non-small cell lung cancer cell LCC168 strain to the WNT inhibitor was shown in (B) of FIG. 6. In the cell structure of the LCC028-3 strain and the JC215 strain (in the figure, “3D”), the relative cell survival rate was higher and the anticancer effect against each anticancer drug was smaller than that of the cells (in the figure, “2D”) 2D-cultured. It was presumed that this was because the ERK/MAPK pathway, the PI3K/AKT pathway, and the Jak-STAT pathway were activated by HGF or VEGF secreted from the stromal cell of the cell structure. On the other hand, in the cell structure of the JC215 strain and the LCC168 strain (in the figure, “3D”), the relative cell survival rate was lower than that of the cells (in the figure, “2D”) 2D-cultured, and high anticancer effect was obtained. It was presumed that this was because the WNT/β-Catenin pathway was activated by HGF secreted from the stromal cell of the cell structure.

What is claimed is:

1. An evaluation method of an anticancer drug, comprising:
culturing a cell structure containing a cancer cell and a stromal cell in a presence of one or two or more of anticancer drugs; and
evaluating an anticancer effect of the anticancer drug using, as an index, the number of cells having a proliferation ability of the cancer cell in the cell structure after the culturing,
wherein a signal transduction pathway in the cancer cell stimulated by a hepatocyte growth factor, a placental growth factor, or a vascular endothelial growth factor is more activated than a signal transduction pathway in a normal cell.

2. The evaluation method of an anticancer drug according to claim 1,
wherein the signal transduction pathway is an ERK/MAPK pathway, a PI3K/AKT pathway, a Jak-STAT pathway, or a WNT/β-Catenin pathway.

3. The evaluation method of an anticancer drug according to claim 1,
wherein the cancer cell is one or more selected from the group consisting of a HER2-positive cancer cell, an ALK-positive cancer cell, a BRAF-positive cancer cell, and a ROS1-positive cancer cell.

4. The evaluation method of an anticancer drug according to claim 1,
wherein the anticancer drug targets a molecule constituting a signal transduction pathway stimulated by a hepatocyte growth factor, a placental growth factor, a vascular endothelial growth factor, or a basic fibroblast growth factor, or a molecule activating the molecule.

5. The evaluation method of an anticancer drug according to claim 4,
wherein the anticancer drug is a HER2 inhibitor, an ALK inhibitor, a BRAF inhibitor, or a WNT inhibitor.

6. The evaluation method of an anticancer drug according to claim 1,
wherein the stromal cell is one or more selected from the group consisting of a fibroblast, a dendritic cell, a macrophage, and a mast cell.

7. The evaluation method of an anticancer drug according to claim 1,
wherein the cell structure has a cancer cell layer on an outer surface.

8. The evaluation method of an anticancer drug according to claim 1,
wherein the cell structure includes, as a cell constituting the stroma, a fibroblast and one or more selected from the group consisting of a vascular endothelial cell and a lymphatic endothelial cell, and
a total number of cells of the vascular endothelial cell and the lymphatic endothelial cell in the cell structure is 0.1% or more of a number of cells of the fibroblast.

9. The evaluation method of an anticancer drug according to claim 1,
wherein a thickness of the cell structure is 5 m or more.

10. The evaluation method of an anticancer drug according to claim 1,
wherein the cell structure includes a vascular network structure.

11. The evaluation method of an anticancer drug according to claim 1,
wherein the cancer cell is a cancer cell collected from a cancer patient.

12. The evaluation method of an anticancer drug according to claim 1,
wherein a culture time in the culture step is 24 to 96 hours.

13. The evaluation method of an anticancer drug according to claim 1, further comprising:
before the culture step, forming the cell structure
wherein the forming the cell structure comprises:
(a) mixing cells, an extracellular matrix component, and a polymer electrolyte in a cationic buffer solution to obtain a mixture;
(b) seeding the mixture obtained in the (a) in a cell culture container; and
(c) obtaining a cell structure in which the cells are laminated in multilayers in the cell culture container after the (b).

14. A selection method of an anticancer drug comprising:
culturing a cell structure containing a cancer cell and a stromal cell in a presence of one or two or more of anticancer drugs; and
selecting an anticancer drug as an anticancer drug having an anticancer effect to the cancer cells when, using the number of cells having a proliferation ability of the cancer cells in the cell structure after the culturing as an index, the number of cells having a proliferation ability of the cancer cells in the cell structure is less than the case where the cell structure is cultured under the condition where the anticancer drug does not exist,
wherein a signal transduction pathway in the cancer cells stimulated by a hepatocyte growth factor, a placental growth factor, a vascular endothelial growth factor, or a basic fibroblast growth factor is more activated than a signal transduction pathway in a normal cell.

15. The selection method of an anticancer drug according to claim 14,
wherein the signal transduction pathway is an ERK/MAPK pathway, a PI3K/AKT pathway, a Jak-STAT pathway, or a WNT/β-Catenin pathway.

16. The selection method of an anticancer drug according to claim 14,
wherein the cancer cell is one or more selected from the group consisting of a HER2-positive cancer cell, an ALK-positive cancer cell, a BRAF-positive cancer cell, and a ROS1-positive cancer cell.

17. The selection method of an anticancer drug according to claim 14,
wherein the anticancer drug targets a molecule constituting a signal transduction pathway stimulated by a hepatocyte growth factor, a placental growth factor, a vascular endothelial growth factor, or a basic fibroblast growth factor, or a molecule activating the molecule.

18. The selection method of an anticancer drug according to claim 16,
wherein the anticancer drug is a HER2 inhibitor, an ALK inhibitor, a BRAF inhibitor, or a WNT inhibitor.

19. The selection method of an anticancer drug according to claim 14,
wherein the cell structure has a cancer cell layer on an outer surface.

20. The selection method of an anticancer drug according to claim 14,
wherein the cancer cell is a cancer cell collected from a cancer patient.
